(19) United States
(12) Patent Application Publication
(10) Pub. No.: US 2023/0015156 A1
(43) Pub. Date: Jan. 19, 2023

(54) ANALYSIS DEVICE, ANALYSIS METHOD, AND RECORDING MEDIUM

(71) Applicant: Konica Minolta, Inc., Tokyo (JP)

(72) Inventors: Hitoshi FUTAMURA, Tokyo (JP); Shinsuke KATSUHARA, Tokyo (JP)

(21) Appl. No.: 17/863,661

(22) Filed: Jul. 13, 2022

(30) Foreign Application Priority Data
Jul. 13, 2021 (JP) 2021-115612

(51) Int. Cl. G16H 50/20; G16H 10/60

(52) U.S. Cl. CPC G16H 50/20; G16H 10/60

(57) ABSTRACT

An analysis device includes a hardware processor, an acquirer, and an outputter, wherein the hardware processor acquires first medically-related information obtained through computer processing performed on medical information, the acquirer acquires second medically-related information created by a user on a basis of the medical information, the hardware processor compares the acquired first medically-related information and the second medically-related information acquired by the acquirer, and the hardware processor outputs statistical information on a basis of the first medically-related information and the second medically-related information.

### CROSS-REFERENCE TO RELATED APPLICATIONS

The present invention claims priority under 35 U.S.C. § 119 to Japanese Application, 2021-115612, filed on Jul. 13, 2021, the entire contents of which being incorporated herein by reference.

### BACKGROUND

### Technological Field

The present invention relates to an analysis device, an analysis method, and a recording medium.

### Description Of The Related Art

In recent years, along with the development of artificial intelligence (AI) technology, analysis by AI has been introduced also to the medical field, and attempts are being made to use AI to support the analysis and diagnosis of medical information such as image diagnosis that has been performed by physicians in the past.

For example, JP 5501491 discloses a diagnostic support device that detects a difference between first medically-related information based on user-created information and second medically-related information obtained through computer processing, and causes a display to display the difference between the two in a display format according to the combination of a lesion name included in the first medically-related information and a lesion name included in the second medically-related information.

In the clinical practice of medicine, it is desirable to perform examinations and diagnoses appropriately and rapidly, and to make diagnosis more efficient and optimized to reduce the burden on physicians.

The introduction of AI analysis is expected to contribute to such more efficient and optimized diagnosis.

### SUMMARY

However, with the technology disclosed in JP 5501491, the diagnostic result from the AI and the diagnostic result from a radiologist are not evaluated or analyzed. For this reason, if there is a difference between the two, for example, it is unclear which can be trusted and to what extent.

Consequently, there is a problem in that it is ultimately necessary to verify both the diagnostic result from the AI and the diagnostic result from the radiologist, and more efficient and optimized diagnosis is not necessarily achieved.

The present invention has been devised in the light of such problems in the technology of the related art described above, and an object of the present invention is to provide an analysis device, an analysis method, and a recording medium that can indicate the degree of reliability of each result efficiently and appropriately in the case where there is a plurality of analysis results or diagnostic results with respect to medical information.

To achieve at least one of the abovementioned objects, according to an aspect of the present invention, an analysis device reflecting one aspect of the present invention includes: an analyzer that acquires first medically-related information obtained through computer processing performed on medical information;
an acquirer that acquires second medically-related information created by a user on a basis of the medical information; and
a comparison processor that compares the first medically-related information acquired by the analyzer and the second medically-related information acquired by the acquirer, wherein
the comparison processor is provided with an outputter that outputs statistical information on a basis of the first medically-related information and the second medically-related information.

According to another aspect,
an analysis method includes:
analyzing medical information through computer processing to acquire first medically-related information;
acquiring second medically-related information created by a user on a basis of the medical information; and
comparing the first medically-related information acquired by the analyzing and the acquired second medically-related information,
   wherein
the comparing includes outputting statistical information on a basis of the first medically-related information and the second medically-related information.

According to another aspect,
a recording medium storing a program
causes a computer to perform:
analyzing medical information through computer processing to acquire first medically-related information;
acquiring second medically-related information created by a user on a basis of the medical information; and
comparing the first medically-related information acquired by the analyzing and the acquired second medically-related information,
   wherein
the comparing includes outputting statistical information on a basis of the first medically-related information and the second medically-related information.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is an overall configuration diagram of a medical imaging system according to the present embodiment;

FIG. 2 is a key block diagram illustrating a functional configuration of an embodiment of an analysis device according to the present invention;

FIG. 3A is an example of statistical information in which an analysis result from an AI and a diagnostic result from a radiologist regarding a diagnosis of a medical image are summarized in a table; FIG. 3B is an example of statistical information in which analysis results from an AI and diagnostic results from a radiologist accumulated over a fixed period are summarized in a table;

FIG. 4 is a flowchart illustrating an analysis process in a first QA pattern;

FIG. 5 is an explanatory diagram schematically illustrating a flow of the analysis process illustrated in FIG. 4;

FIG. 6 is an explanatory diagram schematically illustrating a flow of a modification of the analysis process; and

FIG. 7 is a flowchart illustrating an analysis process in a second QA pattern.

### DETAILED DESCRIPTION OF EMBODIMENTS

Hereinafter, an embodiment of an analysis device, an analysis method, and a recording medium according to the present invention will be described. However, the scope of the invention is not limited to the illustrated examples.

### [Configuration of Medical Imaging System]

An analysis device according to the present embodiment performs analysis and the like of medical information, namely a medical image, in a medical imaging system, for example.

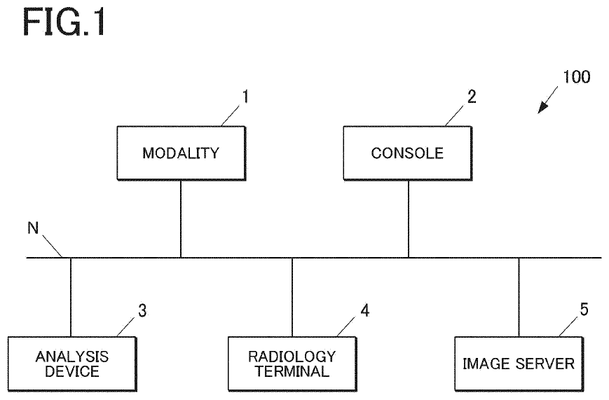

FIG. 1 illustrates a system configuration of the medical imaging system 100.

As illustrated in FIG. 1, the medical imaging system 100 includes a modality 1, a console 2, an analysis device 3, a radiology terminal 4, an image server 5, and the like, the above being connected over a communication network N such as a local area network (LAN), a wide area network (WAN), or the Internet. Each device included in the medical imaging system 100 conforms to the Health Level Seven (HL7) and Digital Image and Communications in Medicine (DICOM) standards, and communication between devices is performed in accordance with HL7 and DICOM. Note that the numbers of the modality 1, the console 2, the radiology terminal 4, and the like are not particularly limited.

The modality 1 is an image generation device such as an X-ray imaging device (DR, CR), an ultrasound diagnostic device (US), a CT, or an MRI, for example, and captures an image of a patient site to be examined as the subject and generates a medical image as medical information on the basis of examination order information transmitted from a radiology information system (RIS) or the like not illustrated. In the medical image generated in the modality 1, supplementary information (such as patient information, examination information, and an image ID) is written to, for example, the header of the image file in accordance with the DICOM standard. The medical image containing supplementary information in this way is transmitted to the analysis device 3 and the radiology terminal 4 through the console 2 or the like.

The console 2 is an imaging control device that controls imaging in the modality 1. The console 2 outputs imaging parameters and image scanning parameters to the modality 1 and acquires the image data of a medical image captured in the modality 1. The console 2 is provided with a controller, display, operating interface, communication interface, storage, and the like which are not illustrated, these components being connected by a bus.

The analysis device 3 is a device that performs any of various types of analysis on the medical image which is medical information. The analysis device 3 is configured as a PC or a mobile terminal, or as a dedicated device. In the present embodiment, a medical image management device such as a picture archiving and communication system (PACS), for example, is included in the analysis device 3.

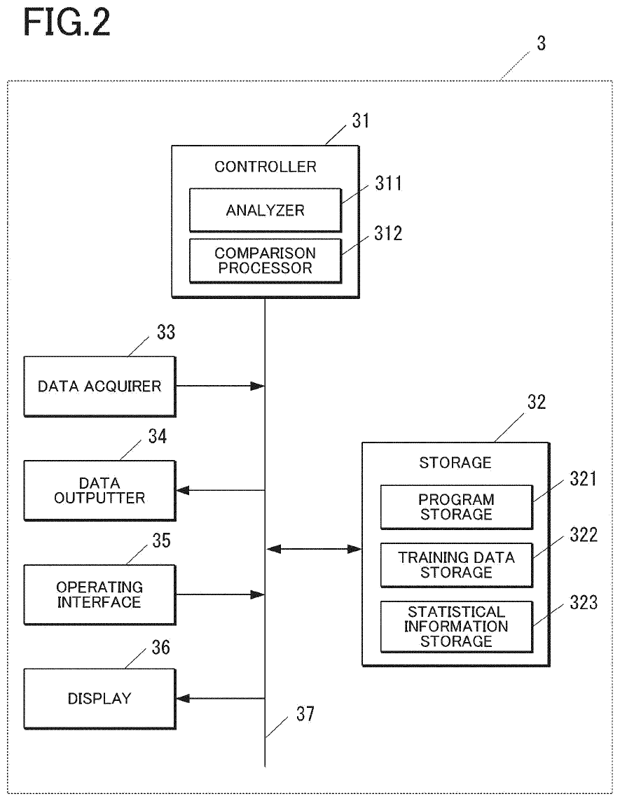

FIG. 2 is a block diagram illustrating a functional configuration of the analysis device 3.

As illustrated in FIG. 2, the analysis device 3 is provided with a controller 31 (hardware processor), storage 32, a data acquirer 33, a data outputter 34, an operating interface 35, a display 36, and the like, these components being connected by a bus 37.

The data acquirer 33 is an acquirer that acquires various data from external devices (such as the console 2 and the radiology terminal 4 described later, for example).

The data acquirer 33 is configured as a network interface, for example, and is configured to receive data from external equipment connected in a wired or wireless manner through the communication network N. Note that in the present embodiment, the data acquirer 33 is configured as a network interface but may also be configured as a port or the like into which USB memory, an SD card, or the like can be inserted.

In the present embodiment, the data acquirer 33 acquires the image data of a medical image from the console 2, for example. Additionally, the data acquirer 33 acquires a diagnostic result (detection result information regarding a lesion that can be read from a medical image) related to a medical image created by a user (such as a physician, for example) on the basis of a medical image which is medical information from the radiology terminal 4, a radiology report which is a radiology result by a radiologist (such as a radiologist who provides a primary interpretation or a secondary interpretation, for example), and the like as “second medically-related information”.

The data outputter 34 is for outputting information processed by the analysis device 3 to an external destination. For example, a network interface for communicating with the radiology terminal 4, the image server 5, or the like, a connector for connecting an external device (such as a display device or a printer not illustrated, for example), or a port for any of various media such as USB memory is applicable as the data outputter 34.

The operating interface 35 is configured as a keyboard provided with various keys, as a pointing device such as a mouse, as a touch panel attached to the display 36, or the like. Through the operating interface 35, the user is able to perform input operations. Specifically, an operation signal input via a key operation on the keyboard, a mouse operation, or a touch operation on the touch panel is output to the controller 31.

The display 36 is provided with a monitor such as a liquid crystal display (LCD) and displays various screens according to instructions in a display signal input from the controller 31. Note that the configuration is not limited to a single monitor, and a plurality of monitors may also be provided.

On the display 36, as described later, statistical information and the like output from the controller 31 (a comparison processor 312 of the controller 31) is displayed as appropriate.

The controller 31 includes a central processing unit (CPU), random access memory (RAM), and the like, and centrally controls operations by the components of the analysis device 3. Specifically, the CPU reads out and loads various processing programs stored in program storage 321 of the storage 32 onto the RAM and executes various processes according to the programs. In the present embodiment, the controller 31 functions as an analyzer 311, a comparison processor 312, and the like through cooperation with the programs.

The analyzer 311 acquires “first medically-related information” by performing computer processing on the medical information. Specifically, lesion detection and analysis processing is performed on a medical image acquired by the data acquirer 33, and one or multiple lesion detection/analysis results are output as the “first medically-related information”. For the computer processing herein, artificial intelligence (AI) analysis using an AI that performs image diagnosis and image analysis, including lesion detection by computer-aided diagnosis (CAD), is used, for example.

The controller 31 also functions as a learner not illustrated that learns correspondences between medical information (in the present embodiment, medical images) and medically-related information (such as names of lesions), for example, and the analyzer 311 obtains the “first medically-related information” through computer processing performed on medical information (a medical image) on the basis of the correspondences between medical information (medical images) and medically-related information learned by the learner.

In other words, for example, a process of detecting/analyzing a lesion from an input medical image is performed by using a machine learning model created by using deep learning or the like to train the model with a large amount of training data (pairs of a medical image in which a lesion appears and a ground truth label (such as a lesion region in the medical images and a diagnostic name of the lesion (the type of lesion))).

The “first medically-related information” acquired in this way is information about the name, location, and the like of a lesion, for example, and is attached to the image data of the medical image as supplementary information.

The comparison processor 312 compares the “first medically-related information” acquired by the analyzer 311 to the “second medically-related information” acquired by the data acquirer 33. In other words, the two sets of information are checked against each other, and a result of the comparison is output.

Specifically, it is clearly indicated whether the “first medically-related information” and the “second medically-related information” agree or do not agree (differ). The comparison of the information presupposes that the comparison processor 312 structures a radiology report or the like created by the user (radiologist) and performs a process of extracting character strings or the like that can be compared to the “first medically-related information” which is the result of the AI analysis. Although omitted from illustration in the drawings, data used to generate structured data, such as dictionary data defining correspondence relationships between character strings and the like, is stored in the storage 32.

In addition, the comparison processor 312 is provided with an outputter that outputs statistical information on the basis of the “first medically-related information” and the “second medically-related information”. In other words, the comparison processor 312 calculates statistical information or the like on the basis of the “first medically-related information” and the “second medically-related information”. The statistical information calculated by the comparison processor 312 is output to the display 36 of the analysis device 3, for example. The display 36 is capable of displaying the output statistical information.

Note that the output destination of the statistical information is not limited to the above, and it is also possible to output the statistical information to the data outputter 34 and display the statistical information on an external display device or any of various types of terminal devices.

The statistical information herein includes numerical values and graphical information obtained by investigating a certain group under fixed conditions such as time and place and combining and processing the results. The statistical information also includes information in which the attributes of a certain group are expressed quantitatively as numerical values or graphs from a distribution of individual components of the group. The statistical information includes a ratio of agreement or non-agreement between medically-related information, and the ratio of agreement or non-agreement between medically-related information includes a correct answer ratio for other medically-related information when prescribed medically-related information is treated as ground truth data, for example.

In the present embodiment, a ratio of agreement or non-agreement as a result of checking the “first medically-related information” and the “second medically-related information” against each other is calculated as the statistical information.

In other words, by treating either one of the “first medically-related information” or the “second medically-related information” as ground truth data and calculating the ratio of agreement or non-agreement for the other data against the ground truth data, the comparison processor 312 can obtain the reliability of the other data with respect to the ground truth data.

For example, the comparison processor 312 treats the “first medically-related information” which is the result of the AI analysis as “first ground truth data” and calculates (outputs) the correct answer ratio of the “second medically-related information” which is the diagnostic result from a radiologist as statistical information on the basis of the “first ground truth data”. This arrangement makes it possible to understand the reliability of the diagnostic result from the radiologist with respect to the AI analysis.

Conversely, the “second medically-related information” which is the diagnostic result from the radiologist may be treated as “second ground truth data”, and the correct answer ratio of the “first medically-related information” which is the result of the AI analysis may be calculated (output) as statistical information on the basis of the “second ground truth data”. This arrangement makes it possible to understand the reliability of the analysis result from the AI with respect to the diagnosis by the radiologist.

Note that the statistical information calculated and output by the comparison processor 312 is not limited to the above.

For example, character strings (keywords) appearing in common in both the “first medically-related information” and the “second medically-related information” may also be extracted as the statistical information. As another example, any of various types of proportions and statistics, such as the degree of scattering of character strings not common to both (such as what kinds of character strings appear in what proportions), for example, may also be extracted as the statistical information.

The comparison processor 312 may also output a table (such as a matrix or heatmap information) summarizing such information in a list of findings as the statistical information.

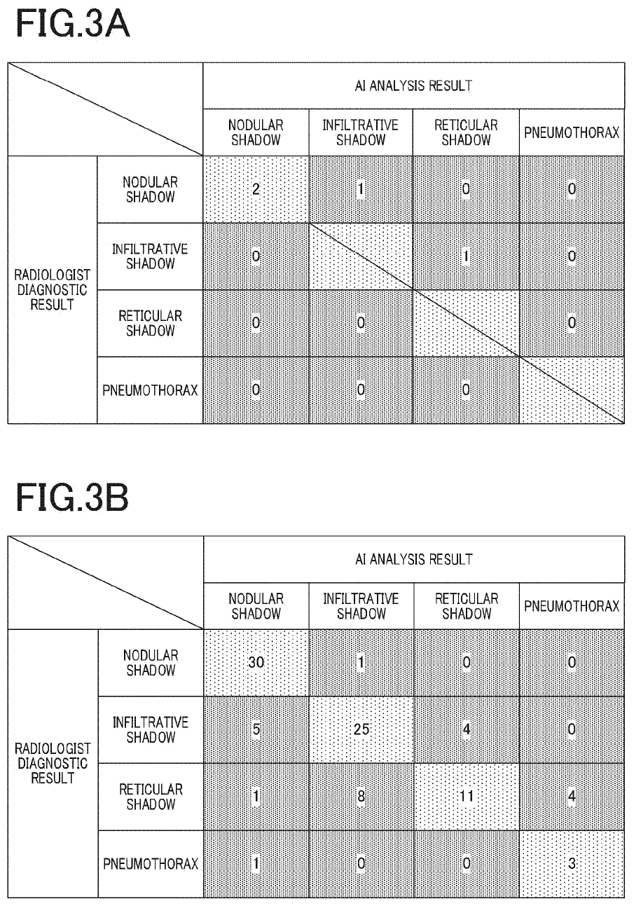

FIG. 3A is an example of statistical information in which the “first medically-related information” which is an analysis result from an AI and the “second medically-related information” which is a diagnostic result from a user (a specialist who provides a primary interpretation or a secondary interpretation) regarding a diagnosis of a medical image are summarized in a table. In FIG. 3A, the analysis result from the AI is indicated on the vertical axis while the diagnostic result from the physician (radiologist) is indicated on the horizontal axis.

From a single medical image, it is possible to read various findings such as the possibility of multiple lesions rather than only a single lesion. For example, FIG. 3A illustrates a case where the medical image subjected to analysis and diagnosis is an X-ray image of the chest. In this case, “nodular shadow”, “infiltrative shadow”, “reticular shadow”, “pneumothorax” and the like are conceivable as findings by the AI or the radiologist. In the examples illustrated in the drawings, the lightly-shaded diagonal line represents items for which the analysis result from the AI and the diagnostic result from the radiologist are in agreement, and in the example of FIG. 3A, a diagnosis of “nodular shadow” for the medical image in question has been accepted by both the AI and the radiologist.

In the examples illustrated in the drawings, “infiltrative shadow” and “reticular shadow” have been accepted in addition to “nodular shadow” in the analysis result from the AI, but in the diagnostic result from the radiologist, only “nodular shadow” is acknowledged, and the others are not acknowledged. By outputting statistical information summarized in a table in this way, it can be made clear which judgments are in agreement and which judgments are not in agreement, whether there is an agreed diagnosis between the AI and the radiologist, and also what kinds of lesions are a possibility from the image.

Also, FIG. 3B is an example of statistical information in which the “first medically-related information” which is the result of the AI analysis and the “second medically-related information” which is a diagnostic result from a user (a specialist who provides a primary interpretation or a secondary interpretation) regarding a diagnosis of a medical image accumulated over a period of some length, such as one week or one month, are summarized in a table.

Like FIG. 3A, FIG. 3B is for the case where the medical images to be analyzed and diagnosed are X-ray images of the chest and illustrates the analysis results from the AI on the vertical axis and the diagnostic results from the physician (radiologist) on the horizontal axis. By taking statistical information over a period of some length, it is possible to see trends such as what kinds of items tend to be in agreement and what kinds of items tend to differ between the analysis results from the AI and the diagnostic results from the radiologist. For example, the ratio at which something that is not “pneumothorax” is judged to be “pneumothorax” is relatively low, but the ratio at which only either one of the AI or the radiologist acknowledges something to be “infiltrative shadow”, or “reticular shadow” is higher, and is conceivably an item that is easily misrecognized by both the AI and the radiologist.

Note that to simplify the illustration, FIG. 3A and FIG. 3B illustrate the four items “nodular shadow”, “infiltrative shadow”, “reticular shadow”, and “pneumothorax” as the items of the findings, and only a limited number is illustrated as the number of findings for each item. However, in actuality, analysis and judgments are made for over a dozen items or the like from a single medical image, for example. In this regard, by illustrating the statistical information summarized in a table like in FIG. 3A and FIG. 3B, the situation of agreement/non-agreement between the analysis result from the AI and the diagnostic result from the radiologist, trends, and the like can be presented in an easily understood way, even when there are many items in the findings and many findings.

In the display 36 which is the output destination of the statistical information output by the comparison processor 312, the statistical information is displayed appropriately according to its content and the like.

For example, the output format (display format) of the statistical information may be changed according to the statistical information (such as the ratio of agreement or the ratio of non-agreement). For example, only items that are in non-agreement may be displayed using a conspicuous color, large text, or the like.

Also, the method for changing the output format (display format) of the statistical information according to the statistical information (such as the ratio of agreement or the ratio of non-agreement) is not limited to changing the color, text, and the like when the statistical information is displayed.

For example, the statistical information may also be output by showing a design such as a mark, and in this case, a threshold value may be set with respect to the statistical information (such as the ratio of agreement or the ratio of non-agreement), for example, such that “Y” is displayed if the statistical information is equal to or greater than a fixed value, and “N” is displayed if the statistical information is less than the fixed value.

Showing of a mark or the like rather than a numeral makes it easy for a physician or the like to grasp the information intuitively, and also has superior visibility. For this reason, in situations where there are many cases to administer (the number of cases for which statistical information is output in the comparison processor 312) in particular, faster and more efficient processing can be achieved by showing a mark or the like.

As another example, the way in which information is displayed on-screen may be changed between the case of a high correct answer ratio in the analysis result from the AI and the case of a low correct answer ratio in the analysis result from the AI with respect to the ground truth data. For example, in the case where the analysis result from the AI has a low correct answer ratio and is not very reliable, the analysis result from the AI and the diagnostic result from the radiologist are both displayed conspicuously for items which are not in agreement between the analysis result from the AI and the diagnostic result from the radiologist. Conversely, in the case where the analysis result from the AI has a high correct answer ratio and is reliable, only the analysis result from the AI may be displayed conspicuously for items not in agreement.

Note that as described above, the statistical information is information that indicates the ratio of agreement/non-agreement between analysis results from the AI and diagnostic results from the radiologist, but the diagnostic results on the radiologist side in this case may be results from the hospital as a whole or results from individual radiologists.

In cases where a variety of statistical information can be calculated by slicing the data differently, such as for the hospital as a whole or for individual radiologists, the way in which the information is displayed, and the like may also be switched for each case.

In the case of calculating the statistical information by taking various slices in this way, if the AI is highly reliable, for example, information indicating the ratio of agreement/non-agreement between diagnostic results from individual radiologists and analysis results from the AI can also be used as a tool when a leader checks the correct answer ratio of a novice radiologist or the like (that is, checks the degree to which the novice radiologist could arrive at the same diagnostic results as the AI).

Also, if the statistical information can be displayed in a filtered way according to the modality 1 or according to examinations by the requested department, it is also possible to verify the correct answer ratio (diagnostic accuracy) according to the modality 1 or the requested department. If such verification is possible, the results can also be used as reference values for improvement measures, such as holding radiology training sessions for departments with a low correct answer ratio, for example.

Furthermore, by segmenting the statistical information calculation range according to the radiology date or the like, it is also possible to use the statistical information as an objective resource when evaluating the degree of improvement, such as evaluating how much the correct answer ratio from 2020 has improved in 2021, for example.

The storage 32 is configured as a hard disk drive (HDD), semiconductor memory, or the like, and stores programs for executing various processes like the process for analyzing medical information such as medical images described later, and parameters, files, and the like necessary for the execution of the programs.

For example, besides program storage 321 that stores various programs, training data storage 322, statistical information storage 323, and the like are stored in the storage 32 of the present embodiment.

The training data storage 322 stores a large amount of training data and a machine learning model created by using deep learning or the like to train the model using the training data as described above, for example.

Also, although not illustrated in the drawings, the storage 32 also stores dictionary data or the like used to generate structured data in which the “second medically-related information”, that is, radiology reports or the like created by a user (radiologist) is structured as described above.

The radiology terminal 4 is a computer device provided with a controller, an operating interface, a display, a storage, and a communication interface, for example, and reads out medical information, namely a medical image, from the image server 5 or the like and displays the image for interpretation.

The user (primary radiologist, secondary radiologist) interprets the medical image on the radiology terminal 4 and creates a radiology report or the like as the “second medically-related information”, which is a diagnostic result from the radiologist in relation to the medical image.

The image server 5 is a server forming a picture archiving and communication system (PACS), for example, and stores patient information (such as a patient ID, patient name, date of birth, age, sex, height, and weight) and examination information (such as an examination ID, examination date and time, type of modality, examination site, requested department, and purpose of examination) for a medical image output from the modality 1, an image ID of the medical image, information about the analysis result from the AI (that is, the “first medically-related information”) output from the analyzer 311 of the analysis device 3 and the “second medically-related information” which is the diagnostic result from the radiologist, such as a radiology report or the like created by the user (radiologist) on the radiology terminal 4, the result (comparison result) of checking the information against each other in the comparison processor 312 of the analysis device 3, the statistical information (like the information illustrated in FIGS. 3A and 3B, for example), and the like in association with each other in a database.

Also, the image server 5 reads out a medical image requested by the radiology terminal 4 and the various supplementary information attached to the medical image from the database and causes the image and information to be displayed on the radiology terminal 4.

### [Analysis Method in Present Embodiment]

The analysis method in the present embodiment includes: analyzing medical information such as a medical image through computer processing to acquire “first medically-related information”; acquiring “second medically-related information” created by a user on the basis of the medical information; and comparing the “first medically-related information” acquired by the analyzing and the acquired “second medically-related information”, wherein the comparing includes outputting statistical information on the basis of the “first medically-related information” and the “second medically-related information”.

According to the analysis method in the present embodiment, by jointly considering the analysis result from the AI and the diagnostic result from the radiologist, quality assurance (hereinafter referred to as “QA”) can be performed for the diagnostic accuracy in relation to medical information (a medical image).

With regard to QA patterns, the flow of the analysis method is different depending on the degree to which the analysis result from the AI can be trusted.

A first QA pattern is a flow adopted in the case where the analysis result from the AI is not very reliable, and in the case where AI analysis cannot be trusted completely.

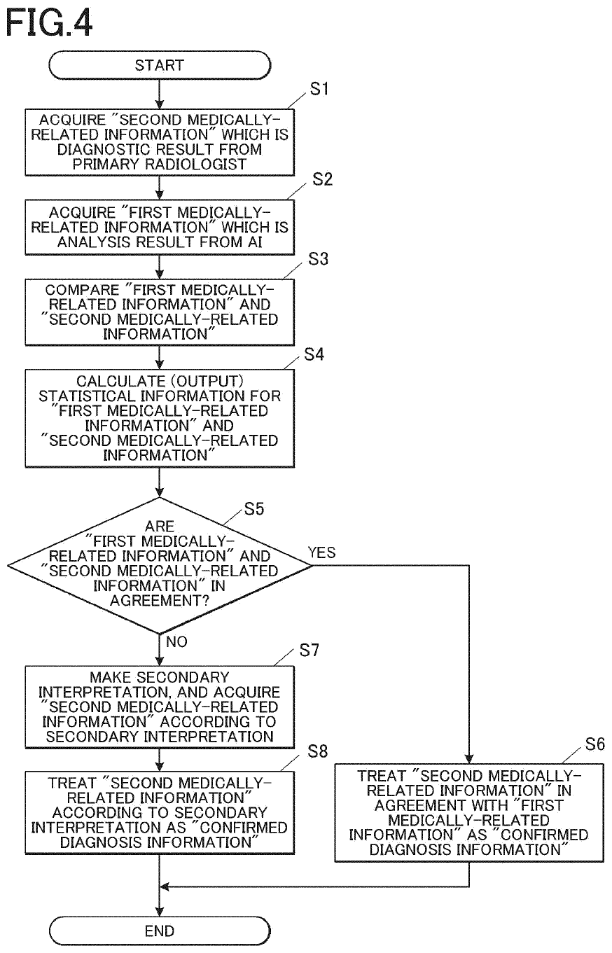

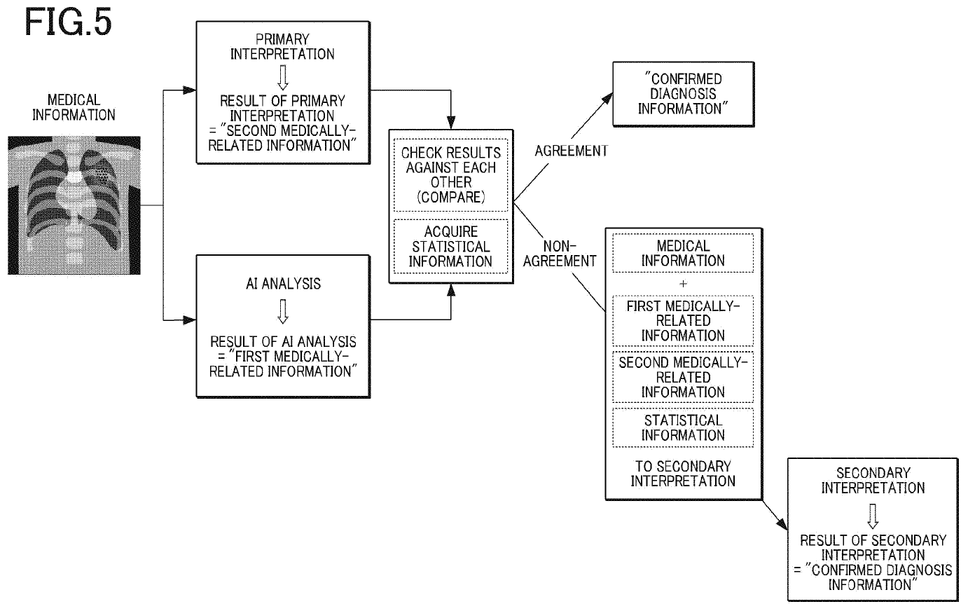

FIG. 4 is a flowchart illustrating an analysis process in the first QA pattern, and FIG. 5 is an explanatory diagram schematically illustrating the flow of processes.

As illustrated in FIG. 4 and FIG. 5, in the first QA pattern, first, “second medically-related information (according to the primary interpretation)” which is a diagnostic result from a primary radiologist, such as a radiology report or the like created by having a radiologist who provides a primary interpretation interpret the medical information (herein, a medical image) to be processed, is acquired by the data acquirer 33 (step S1; acquiring).

Also, the medical information (medical image) to be processed is analyzed by an AI (AI application) in the analyzer 311 of the analysis device 3, and “first medically-related information” which is the analysis result (diagnostic result) is acquired (step S2; analyzing).

Thereafter, the “first medically-related information” and the “second medically-related information” are checked against each other (compared) in the comparison processor 312 (step S3; comparing).

Also, in the present embodiment, statistical information is calculated and output as appropriate by the comparison processor 312 (step S4; outputting). The kind of information to be output as the statistical information may be set as appropriate according to the type of medical image (such as which part of the body was imaged to obtain the image), the reliability of the analysis result from the AI, and the like. The user (such as a primary radiologist or a secondary radiologist) may also choose what kind of statistical information is to be calculated and output.

In the comparison processor 312, as a result of checking (comparing) the “first medically-related information” and the “second medically-related information” against each other, it is determined whether the two are in agreement (step S5). If the information is in agreement (step S5; YES), the diagnostic result and analysis result are treated as information about a confirmed diagnosis (hereinafter referred to as “confirmed diagnosis information”) (step S6).

On the other hand, if the information is not in agreement (the case where the information differs, step S5; NO), the medical information is passed to a secondary radiologist (final radiologist) who provides a secondary interpretation (step S7). Thereafter, the result from the secondary interpretation is treated as the “confirmed diagnosis information” (step S8). When the medical information is passed to secondary interpretation, as illustrated in FIG. 5, the medical information (medical image) to be processed, the “first medically-related information”, the “second medically-related information”, and the statistical information calculated and output on the basis of the “first medically-related information” and the “second medically-related information” are sent to the radiology terminal 4 operated by the secondary radiologist, the secondary radiologist refers to the information to make a secondary interpretation, and creates a diagnostic result such as a radiology report as “second medically-related information (according to the secondary interpretation)”.

In this way, in the process illustrated in FIG. 4 and FIG. 5, the “second medically-related information (according to the primary interpretation)” which is the diagnostic result from the primary radiologist and the “first medically-related information” which is the analysis result (diagnostic result) from the AI are checked against each other (compared), and the result (check result and statistical information) is sent to the secondary radiologist.

With this arrangement, the secondary radiologist can refer to this information when making the secondary interpretation and scrutinize only the portions of the results where the conclusions differ, for example. Consequently, an efficient and appropriate interpretation can be provided.

In particular, in the case of creatively designing how the statistical information is displayed, such as by changing the color or the like to emphasize the portions where the conclusions differ between the “first medically-related information” and the “second medically-related information”, and touching an item or the like for which the conclusions are different to apply a mark or change the color for emphasis with respect to a portion acknowledged to have an abnormality related to the item in the medical image, the efficiency and accuracy of the interpretation (secondary interpretation) can be improved further.

When “confirmed diagnosis information” is obtained for the medical image treated as the medical information, the medical image is stored in the image server 5 in association with the “confirmed diagnosis information” confirmed in the analysis device 3.

Note that the save locations for the medical image, the various information attached thereto (such as the “first medically-related information”, the “second medically-related information”, and the statistical information), the “confirmed diagnosis information”, and the like are not particularly limited.

For example, in the case where the analysis device 3, the radiology terminal 4, and the image server 5 form a PACS, the above information may be saved and managed collectively in an information management server or the like on the PACS.

Moreover, not all of the above information has to be saved, and for example, only the medical image and the “confirmed diagnosis information” may be saved as a set.

Note that the “confirmed diagnosis information” referred to herein is a diagnostic result obtained by having a specialist in radiology make a definite conclusion from the medical image and the findings and analysis result obtained on the basis thereof.

The final diagnosis of the patient is made by a clinician comprehensively considering the “confirmed diagnosis information” for the medical image in addition to examination data, observation data, and the like obtained through various examinations, observations, and so on.

Note that FIG. 4 and FIG. 5 illustrate the example of a case where either one of the “second medically-related information” according to the primary interpretation or the “first medically-related information” according to the AI analysis is treated as ground truth data to calculate the reliability of the other with respect to the ground truth data as the statistical information, but the target of comparison when calculating the reliability is not limited to the “second medically-related information (according to the primary interpretation)” and the “first medically-related information”.

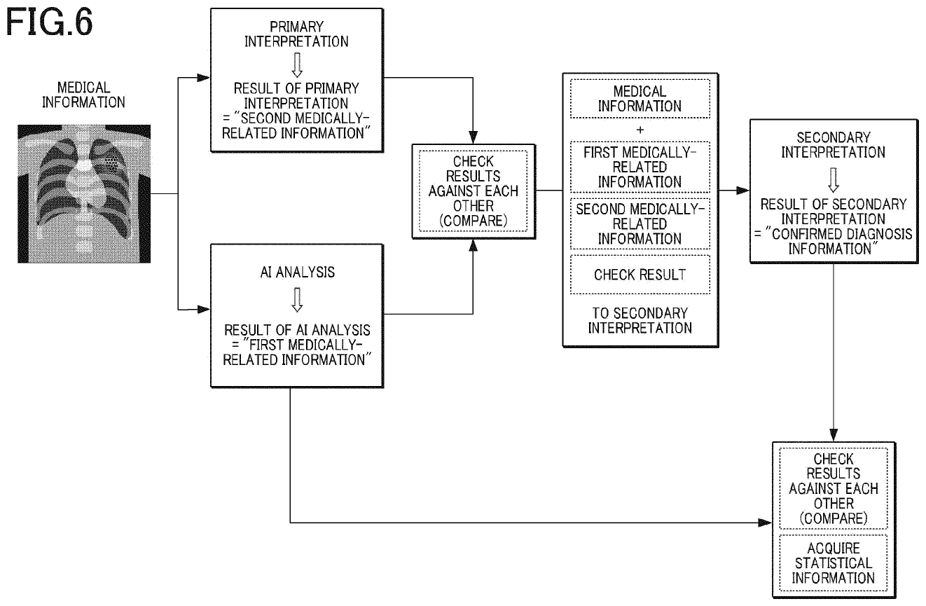

For example, as illustrated in FIG. 6, the comparison processor 312 may also treat either one of the “first medically-related information” from the AI analysis or the “second medically-related information (according to the secondary interpretation)” (“confirmed diagnosis information”) which is the diagnostic result from the secondary radiologist as ground truth data, and calculate (output) the ratio of agreement or non-agreement of the other data with respect to the ground truth data as the statistical information.

In other words, for example, the comparison processor 312 may treat the “second medically-related information (according to the secondary interpretation)” (“confirmed diagnosis information”) which is the diagnostic result from the secondary radiologist as “second ground truth data”, and calculate (output) the correct answer ratio of the “first medically-related information” from the AI analysis on the basis of the “confirmed diagnosis information” as the statistical information. With this arrangement, the reliability of the AI analysis with respect to the diagnostic result from the secondary radiologist (“confirmed diagnosis information”) can be known.

Conversely, the “first medically-related information” which is the result of the AI analysis may be treated as the “second ground truth data”, and the correct answer ratio of the “second medically-related information (according to the secondary interpretation)” (“confirmed diagnosis information”) which is the diagnostic result from the secondary radiologist may be calculated (output) on the basis of the “second ground truth data” as the statistical information. With this arrangement, the reliability of the diagnosis by the secondary radiologist with respect to the AI analysis result can be known.

Note that in the case where either one of the “first medically-related information” or the “second medically-related information (according to the secondary interpretation)” (“confirmed diagnosis information”) is treated as ground truth data to calculate the ratio of agreement or non-agreement between the two in this way, as illustrated in FIG. 6, the “second medically-related information (according to the primary interpretation)” as the result of the primary interpretation is only checked against the “first medically-related information” (in other words, statistical information such as the ratio of agreement is not calculated), and the medical information (medical image) to be processed, the “first medically-related information”, the “second medically-related information”, and check information (agreement/non-agreement information) obtained by checking the “first medically-related information” and the “second medically-related information” against each other are sent to the radiology terminal 4 operated by the secondary radiologist. Thereafter, the secondary radiologist refers to this information to make a secondary interpretation and creates a diagnostic result such as a radiology report as the “second medically-related information (according to the secondary interpretation)”, which is treated as the “confirmed diagnosis information”.

Note that, as illustrated in FIG. 6, in the case where the result of checking the “first medically-related information” from the AI analysis and the “second medically-related information (according to the primary interpretation)” against each other is passed on for a secondary interpretation when the two are in agreement and when the two are not in agreement, without branching the process, the statistical information (such as the ratio of agreement or non-agreement, for example) may be calculated only between the “first medically-related information” and the “second medically-related information (according to the secondary interpretation)” (“confirmed diagnosis information”), or between both the “first medically-related information” and the “second medically-related information (according to the primary interpretation)” and, the “first medically-related information” and the “second medically-related information (according to the secondary interpretation)” (“confirmed diagnosis information”).

Furthermore, in the case of comparing the “first medically-related information” from the AI analysis and the “second medically-related information (according to the secondary interpretation)” which is the diagnostic result from the secondary radiologist (in other words, the “confirmed diagnosis information”), the comparison processor 312 may only make a comparison by checking the two against each other, without calculating statistical information. This arrangement can bring image diagnosis to a close even more rapidly.

In this case, too, check information that indicates whether the diagnostic result from the secondary radiologist (final radiologist) is in agreement with the analysis result from the AI as a result of checking the two against each other is attached to the “confirmed diagnosis information”, and therefore a useful decision-making resource can be provided to the clinician who makes a final diagnosis by comprehensively considering the results of image diagnosis, various examination information, and the like.

Note that, unlike the above, in cases where the reliability of the analysis result from the AI is remarkably high or the AI analysis can be trusted completely, the flow of a second QA pattern is adopted.

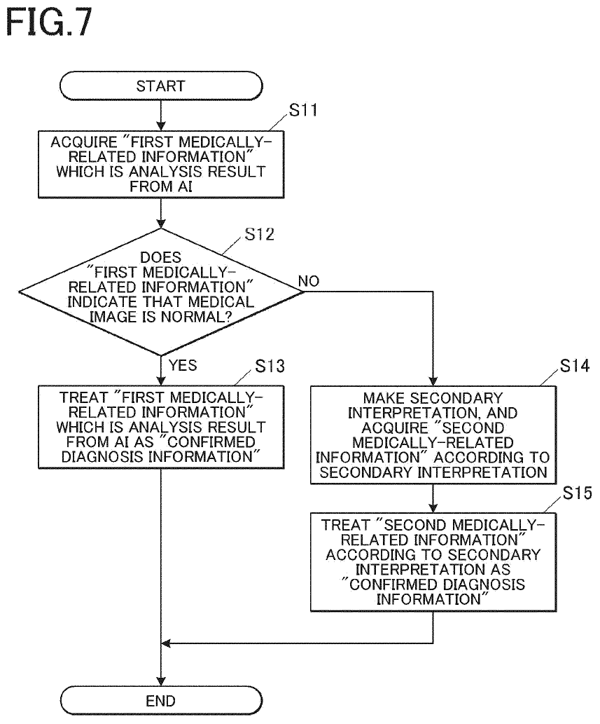

FIG. 7 is a flowchart illustrating an analysis process in the second QA pattern.

As illustrated in FIG. 7, in the second QA pattern, first, the medical information (medical image) to be processed is analyzed by an AI in the analyzer 311 of the analysis device 3, and “first medically-related information” which is the analysis result (diagnostic result) is acquired (step S11).

Note that in this case, the acquisition of “second medically-related information (according to the primary interpretation)” which is the diagnostic result from the primary radiologist is not essential.

Thereafter, the controller 31 determines whether the “first medically-related information” indicates that the medical image is normal (step S12). In other words, it is determined whether the “first medically-related information” includes some kind of abnormal finding with regard to the medical image.

If the “first medically-related information” indicates that the medical image is normal (step S12; YES), the “first medically-related information” which is the diagnostic result from the AI is treated as “confirmed diagnosis information” (step S13).

In this case, the radiologist can be saved from the work of interpreting medical images that the AI has concluded to be normal, the burden on the radiologist can be reduced, and efficient image diagnosis can be performed.

Note that even if the “first medically-related information” indicates that the medical image is normal, this result may not be treated as the “confirmed diagnosis information” immediately, and instead, a “normal label” indicating that the medical image is normal and does not include an abnormal finding may be attached to the “first medically-related information” which is the analysis result from the AI or the like, the medical image to be determined and the “first medically-related information” may be passed to a secondary radiologist (or a final radiologist), and a diagnostic result from the secondary radiologist may be treated as the “confirmed diagnosis information”.

In this case, a diagnosis by the secondary radiologist (final radiologist) is still respected, but by attaching the “normal label”, the burden on the radiologist can be reduced.

On the other hand, in the case where the “first medically-related information” does not indicate that the medical image is normal (the case where an abnormal finding is included, step S12; NO), the medical image to be determined and the “first medically-related information” are passed to the secondary radiologist (or final radiologist) who provides a secondary interpretation (step S14). Thereafter, the diagnostic result from the secondary radiologist is treated as the “confirmed diagnosis information” (step S15).

In this case, too, the comparison processor 312 may check the “first medically-related information” which is the analysis result from the AI and the “confirmed diagnosis information” which is the diagnostic result from the secondary radiologist against each other and calculate (output) statistical information such as the ratio of agreement or non-agreement. When statistical information is output, the statistical information may also be stored in the image server 5 or the like together with the medical image and the “confirmed diagnosis information”. By storing the statistical information in association with the medical image and the “confirmed diagnosis information”, the information can be referred to later when a final diagnosis is made by the clinician or the like.

### [Effects]

As described above, the analysis device 3 according to the present embodiment is provided with the analyzer 311 that acquires the “first medically-related information” obtained through computer processing (namely, AI analysis) performed on medical information, the data acquirer 33 that acquires the “second medically-related information” created by a user (namely, a radiologist who provides a primary interpretation or a secondary interpretation) on the basis of the medical information, and the comparison processor 312 that compares the “first medically-related information” acquired by the analyzer 311 and the “second medically-related information” acquired by the data acquirer 33.

By comparing the diagnosis by the radiologist to the analysis result from the AI, a more careful diagnosis can be made, and appropriate image diagnosis can be performed. Particularly, in cases such as where the object of comparison with the analysis result from the AI is a diagnosis (confirmed diagnosis) by a secondary radiologist, the conclusion reached by a human physician can be verified dispassionately, and more appropriate image diagnosis can be achieved.

Additionally, in the analysis device 3 provided with the analyzer 311 that acquires the “first medically-related information” obtained through computer processing (namely, AI analysis) performed on medical information, the data acquirer 33 that acquires the “second medically-related information” created by a user (namely, a radiologist who provides a primary interpretation or a secondary interpretation) on the basis of the medical information, and the comparison processor 312 that compares the “first medically-related information” acquired by the analyzer 311 and the “second medically-related information” acquired by the data acquirer 33, the comparison processor 312 may also be provided with an outputter that outputs statistical information on the basis of the “first medically-related information” and the “second medically-related information”.

When AI analysis is introduced into image diagnosis, a plurality of analysis results and diagnostic results regarding a medical image which is medical information, such as medically-related information obtained by the AI analysis and medically-related information created by a radiologist who provides a primary interpretation or a secondary interpretation, is obtained. In this case, if the reliability of each result cannot be verified, it is difficult to determine which result should be used as the basis for making a final confirmed diagnosis, and efficient interpretation cannot be achieved.

In this regard, by outputting statistical information such as the ratio of agreement or non-agreement for each result, the degree of reliability can be indicated effectively, and an appropriate confirmed diagnosis can be made efficiently for the medical image which is medical information.

Moreover, if the statistical information is the ratio of agreement or non-agreement between the “first medically-related information” and the “second medically-related information”, the degree to which the analysis result from the AI is in agreement with the diagnostic result from the radiologist can be known.

In cases such as where the analysis result from the AI and the diagnostic result from the radiologist who is a specialist in radiographic interpretation differ greatly, there is a possibility that the results are not very reliable. In this way, an indicator related to reliability can be obtained, and an appropriate diagnosis can be made for the medical image which is medical information.

Also, the comparison processor 312 treats the “first medically-related information” as “first ground truth data”, and outputs the correct answer ratio of the “second medically-related information” as statistical information on the basis of the “first ground truth data”.

Whether the analysis result from the AI or the diagnostic result from the radiologist is correct or not depends on which determination result is treated as the ground truth.

In this regard, by treating the “first medically-related information” obtained by the AI analysis as ground truth data, the reliability of the diagnostic result from the radiologist with respect to the ground truth data can be calculated.

Also, the comparison processor 312 treats the “second medically-related information” as “second ground truth data”, and outputs the correct answer ratio of the “first medically-related information” as statistical information on the basis of the “second ground truth data”.

By treating the “second medically-related information” which is the diagnostic result from the radiologist as ground truth data in this way, the reliability of the analysis result by the AI with respect to the ground truth data can be calculated.

The “second medically-related information” may also be the “confirmed diagnosis information” which is the diagnostic result from the secondary radiologist (or final radiologist).

In this case, by outputting statistical information on the basis of the “first medically-related information” and the “second medically-related information”, the reliability can be obtained for the “confirmed diagnosis information” as the “second medically-related information”.

Also, in the case where the comparison processor 312 treats the “second medically-related information” which is the “confirmed diagnosis information” as “second ground truth data” and outputs the correct answer ratio of the “first medically-related information” as statistical information on the basis of the “second ground truth data”, the reliability of the analysis result from the AI with respect to the “confirmed diagnosis information” can be calculated.

Additionally, the analyzer 311 or the controller 31 including the analyzer 311 is provided with a learner that learns correspondences between medical information (for example, medical images) and medically-related information (such as names of lesions, for example), and the analyzer 311 obtains the “first medically-related information” through computer processing performed on medical information on the basis of the correspondences between medical information and medically-related information learned by the learner.

In the case where the analyzer 311 uses a model obtained through machine learning in this way, the analysis accuracy can be improved through accumulated learning, making it possible to obtain more reliable “first medically-related information”.

### [Modifications]

Note that although an embodiment of the present invention has been described above, the present invention is not limited to such an embodiment, and obviously various modifications are possible within a scope that does not depart from the gist of the present invention.

For example, the above embodiment illustrates an example of a case where the medical information to be analyzed by the analysis device is a medical image, but the medical information is not limited to a medical “image”.

Information or the like acquired by various examinations of a patient may be broadly included in the medical information, and for example, results obtained from any of various types of examinations, such as electrocardiogram waveform data and cardiac sound data, data related to blood flow, and the like may be included in the medical information.

Also, the above embodiment illustrates an example of a case where the analyzer 311 acquires the “first medically-related information” obtained through computer processing (namely, AI analysis) performed on medical information, and the comparison processor 312 may compare the “first medically-related information” to “second medically-related information” created by a user (namely, a radiologist who provides a primary interpretation or a secondary interpretation) on the basis of the medical information and calculate (output) statistical information such as the ratio of agreement or non-agreement between the two, but the target of comparison or the like by the comparison processor 312 is not limited to the above.

For example, the analyzer 311 may also analyze (perform computer processing on) medical information using an AI different from the AI from which the “first medically-related information” was acquired, and thereby acquire “third medically-related information”. In this case, the comparison processor 312 may compare the “first medically-related information” which is the analysis result from one AI to the “third medically-related information” which is the analysis result from a different AI.

Moreover, the comparison processor 312 may treat the “third medically-related information” as “third ground truth data” and calculate (output) the correct answer ratio of the “first medically-related information” as statistical information on the basis of the “third ground truth data”. In this case, the reliability can be compared between Ms.

For example, by seeing how much an AI newly introduced at a certain facility can output the same analysis results as the analysis results (“third medically-related information” which is “third ground truth data”) from a different AI that is already highly reliable for analysis (in other words, by seeing how high of a correct answer ratio can be achieved), the reliability of the newly introduced AI can be calculated.

Also, as described above, the above embodiment illustrates an example of a case where the target of comparison or the like by the comparison processor 312 is the “second medically-related information” which is a diagnostic result from a user (namely, a radiologist) and the “first medically-related information” which is the analysis result from an AI, but the target of comparison or the like by the comparison processor 312 is not limited to the above.

For example, the data acquirer 33 may also acquire “fourth medically-related information” which is a diagnostic result from a primary radiologist and “fifth medically-related information” which is a diagnostic result from a secondary radiologist on the basis of medical information, and the comparison processor 312 may compare the “fourth medically-related information” and the “fifth medically-related information” and calculate (output) statistical information such as the ratio of agreement or non-agreement between the two.

In this case, too, either one of the “fourth medically-related information” or the “fifth medically-related information” can be treated as ground truth data to calculate the reliability of the other.

Also, in the above embodiment, the analysis device 3, the radiology terminal 4, and the image server 5 are illustrated as respectively separate devices in FIG. 1, but the analysis device 3 and the image server 5, or the analysis device 3, the radiology terminal 4, and the image server 5, may also be configured as a single device or a single system.

Note that the present invention obviously is not limited to the above embodiment, modifications, and the like, and alterations can be made, as appropriate, without departing from the gist of the present invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. An analysis device comprising:
a hardware processor;
an acquirer; and
an outputter, wherein
the hardware processor acquires first medically-related information obtained through computer processing performed on medical information,
the acquirer acquires second medically-related information created by a user on a basis of the medical information,
the hardware processor compares the acquired first medically-related information and the second medically-related information acquired by the acquirer, and
the hardware processor outputs statistical information on a basis of the first medically-related information and the second medically-related information.

2. The analysis device according to claim 1, wherein the statistical information is a ratio of agreement or a ratio of non-agreement between the first medically-related information and the second medically-related information.

3. The analysis device according to claim 1, wherein
the hardware processor treats the first medically-related information as first ground truth data, and
outputs a correct answer ratio of the second medically-related information as statistical information on a basis of the first ground truth data.

4. The analysis device according to claim 1, wherein
the hardware processor treats the second medically-related information as second ground truth data, and
outputs a correct answer ratio of the first medically-related information as statistical information on a basis of the second ground truth data.

5. The analysis device according to claim 1, wherein
the hardware processor acquires third medically-related information obtained through computer processing performed on the medical information,
the hardware processor treats the third medically-related information as third ground truth data, and
outputs a correct answer ratio of the first medically-related information as statistical information on a basis of the third ground truth data.

6. The analysis device according to claim 1, wherein the second medically-related information is confirmed diagnosis information.

7. The analysis device according to claim 1, wherein
the second medically-related information is confirmed diagnosis information,
the hardware processor treats the second medically-related information as second ground truth data, and
outputs a correct answer ratio of the first medically-related information as statistical information on a basis of the second ground truth data.

8. The analysis device according to claim 1, further comprising:
a learner that learns correspondences between medical information and medically-related information, wherein
the hardware processor obtains the first medically-related information through computer processing performed on the medical information on a basis of correspondences between medical information and medically-related information learned by the learner.

9. An analysis device comprising:
a hardware processor that acquires first medically-related information obtained through computer processing performed on medical information; and
an acquirer that acquires confirmed diagnosis information created by a user on a basis of the medical information, wherein
the hardware processor compares the acquired first medically-related information and the confirmed diagnosis information acquired by the acquirer.

10. An analysis method comprising:
analyzing medical information through computer processing to acquire first medically-related information;
acquiring second medically-related information created by a user on a basis of the medical information; and
comparing the first medically-related information acquired by the analyzing and the acquired second medically-related information, wherein
the comparing includes outputting statistical information on a basis of the first medically-related information and the second medically-related information.

11. A non-transitory computer readable storage medium storing a program causing a computer to perform:
analyzing medical information through computer processing to acquire first medically-related information;
acquiring second medically-related information created by a user on a basis of the medical information; and
comparing the first medically-related information acquired by the analyzing and the acquired second medically-related information, wherein
the comparing includes outputting statistical information on a basis of the first medically-related information and the second medically-related information.
